(19) United States
(12) Patent Application Publication
(10) Pub. No.: US 2017/0165371 A1
(43) Pub. Date: Jun. 15, 2017

(54) NOVEL SYNTHESIS OF POTENTIAL ESTER PRODRUGS

(71) Applicant: Joel Steven Goldberg, Hillsborough, NC (US)

(72) Inventor: Joel Steven Goldberg, Hillsborough, NC (US)

(21) Appl. No.: 15/438,955

(22) Filed: Feb. 22, 2017

(51) Int. Cl. C07K 1/113; A61K 31/197; C08G 63/91; A61K 38/08; A61K 31/765; C07J 41/00

(52) U.S. Cl. CPC A61K 47/48123; A61K 38/08; A61K 31/197; A61K 31/765; C08G 63/912

(57) ABSTRACT

Esters prodrugs that cross the blood brain barrier can be ideal drugs for treatment of diseases of the central nervous system because the cerebral spinal fluid contains an abundance of esterases. The prodrug can be hydrolyzed into an active drug and a metabolite such as cholesterol that is known to be non-toxic and is familiar to the central nervous system. This invention describes a modification of the Fischer-Speier or Fischer esterification reaction in which one reagent is lipophilic and the other reagent is hydrophilic. The reaction occurs in a heterogeneous mixture. The preferred catalyst is 1.0 M hydrochloric acid and the preferred solvent is acetone. The presence of ester synthesis was confirmed by the hydroxamic acid-ferric perchlorate reaction. The synthesis can be conducted without chemical scaffolds and without protecting functional groups.

### CROSS-REFERENCES TO RELATED APPLICATIONS

None

### FEDERALLY FUNDED RESEARCH

Not applicable

### BACKGROUND OF THE INVENTION

The Fischer-Speier or Fischer esterification reaction is a common method used to synthesize esters from reagents of carboxylic acids and primary or secondary alcohols. Discovered in 1895, esters are formed when an alcohol and carboxylic acid are refluxed in an acid milieu. In the mechanism of the reaction the acid serves as a catalysts and combines to form water as a leaving group. (FIG. 1)

Problems associated with the Fischer esterification reaction include:

1. The reaction rate may be slow.

2. Esterification may be reversible because equilibrium constants of the intermediate reactions only slightly favor product formation.

3. Esters may be synthesized that have a lower boiling point than reactants and therefore may be difficult to isolate.

4. An alcohol, one reagent, is usually the solvent and a reactant and the second reagent requires solubility in alcohol.

5. Protecting groups are required to decrease side reactions formed during reflux heating in the acidic milieu.

6. Hydrophilic and lipophilic reagents may not react because of differences in solubility and a scaffold approach to synthesis may be required.

7. Yields of product may be low.

Esters can be important prodrugs especially those that cross the human blood brain barrier (BBB). Many esterases exist in the cerebral spinal fluid (CSF) that can hydrolyze an ester prodrug into an active drug and products that are familiar and non-toxic to the central nervous system (CNS).

Even though many esterases are present in human plasma, ester prodrugs can still target the CNS. When an ester prodrug is administered, complete degradation of the ester prodrug may not occur during a circulation time so some ester prodrug can cross the BBB. Supporting this observation is clinical experience that intravenous administration of ester drugs such as 2-chloroprocaine, tetracaine, meperidine and cocaine are associated with CNS effects.

Shashoua et al. showed that esters of gamma amino butyric acid (GABA) could be conjugated with cholesterol or linoleic, and these compounds would cross the BBB and undergo hydrolysis in the CNS of mice.(Jacob, Hesse, & Shashoua, 1990; Shashoua, 1991) After the ¹⁴C GABA esters were administered, the ¹⁴C GABA was recovered from the brain and a brain penetration index (BPI) was calculated as the concentration of labeled GABA in the brain divided by the concentration of labeled GABA in the liver. The maximum BPI for the cholesteryl ester of GABA and linoleic acid ester of GABA were 86% and 75% respectively.(Jacob et al., 1990)

In one instance, the synthesis of the butyl ester of GABA was accomplished through a Fischer esterification reaction without a protecting group with n-butanol as a solvent. However GABA is soluble in n-butanol so the mixture was homogenous when compared the heterogeneous mixtures in this invention. Other synthetic methods of GABA esters included protection of the GABA amine and condensation of the protected GABA with an anhydride to synthesize the GABA ester. This synthesis was complex requiring multiple steps. (Jacob, Hesse, & Shashoua, 1987)

Present techniques to synthesize esters from lipophilic and hydrophilic reagents are to scaffold a reagent. The scaffold can be surface-modified cellulose nanocrystals or multi-walled carbon nanotubes.(Abuilaiwi, Laoui, Al-Harthi, & Atieh, 2010; S. M. Spinella et al., 2014; S. Spinella et al., 2016)

Another method to solve reagent solubility problems is to incorporate acidic ionic liquids into the esterification process which serve both as a solvent and acidic catalyst.(Cole et al., 2002; Forbes & Weaver, 2004; Joseph, Sahoo, & Halligudi, 2005)

Conjugating an active drug that may be hydrophilic to a lipophilic molecule to synthesize an ester prodrug is one method to transport medications across the BBB where they can be hydrolyzed into active drug. Since the cost and time required to synthesize ester prodrugs is significant, having a simplified method to synthesize d ester medications capable of crossing the BBB as described in this invention would be very useful.

### DESCRIPTION OF THE DRAWINGS

FIG. 1 shows the mechanism of the Fischer esterification reaction.

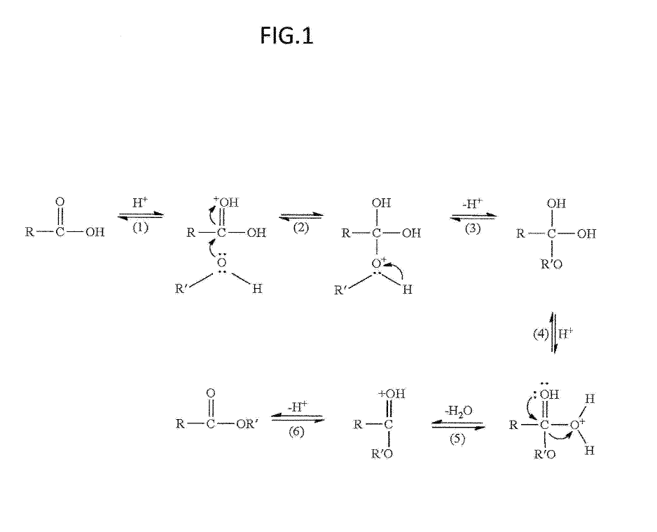

FIG. 2 shows the mechanism of the detection of esters by forming hydroxamic acid and colorimetric detection with ferric perchlorate indicator.

### DETAILED DESCRIPTION OF THE INVENTION

This invention is a novel, simple and improved method to synthesize select ester prodrugs. The prodrugs are synthesized by modification of the 1895 Fisher esterification reaction.

Synthesizing ester prodrugs to permeate the BBB requires that the prodrug be made lipophilic. This can be accomplished by conjugation of the drug with cholesterol or fatty acids such as linoleic or palmitic acids. In the past the synthesis of such prodrugs required protecting groups and synthesis of an anhydride to form the ester. In this invention it was discovered that select ester prodrugs can be synthesized by a simple modification of the Fischer esterification reaction. In the Fischer esterification reaction the alcohol conjugate and alcohol solvent are usually the same compound. In this invention the ester prodrug can be synthesized by combining the active drug that may be a peptide such as leu-enkephalin, a polymer such as oligorners of poly L-lactic acid (PLLA) or poly D-lactic acid (PDLA) or an aminoacid such as GABA that may not be soluble in most organic solvents but soluble in water with a lipophilic carrier such as cholesterol to produce a prodrug.

Cholesterol is essentially insoluble in water but soluble in acetone. In this invention it was discovered that concentrated aqueous solutions of the hydrophilic reagent drug could be refluxed with cholesterol dissolved in acetone to produce an ester prodrug. The yields may be sufficient for lead drug investigations.

Since the Fisher esterification reaction is an equilibrium reaction increasing the concentration of one of the reagents is required to favor formation of products. When the hydrophilic reagent is in excess (two to four times the concentration of the lipophilic reagent) subsequent extraction of the solid end products of the esterification reaction with ether and water will separate the excess hydrophilic reagent.

In this invention it was also discovered that protecting groups were not required for synthesis of selected ester prodrugs. Leu-enkephalin is a penta-peptide composed of amino acids with only a few functional groups. The terminal amine of the peptide and hydroxyl of tyrosine could side react but it was observed that protecting these groups were not needed to form the cholesteryl ester of leu-enkephalin. Similarly the conjugation of GABA with cholesterol to form the ester reaction does not require protection of the GABA amine group. With only a carboxylic acid functional group, esterification of oligomers of PLLA or PDLA requires no protection. Although the cholesteryl ester of PLLA is unlikely to have significant pharmacologic properties, the cholesteryl ester of PDLA may have important CNS drug properties because PDLA is known to sequester L-lactate. (Goldberg, 2016)

### Benefits to Society

The time and cost to synthesizing lead drugs continues to increase. Modification of a known esterification reaction as described in this invention may promote the development of ester prodrugs especially those which may cross the BBB and treat diseases of the CNS.

### Experimental Section

### Synthesis of Ester Prodrugs

### Synthesis of the Cholesteryl Ester of GABA

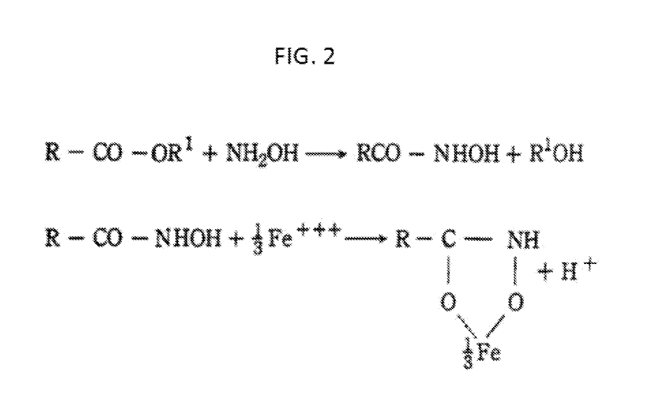

1. Ten microliters of 1.0 M hydrochloric acid was dissolved in a solution of 25 ml of acetone containing 1 millimole or 386 mg of cholesterol. A solution containing 3 millimoles or 309 mg of GABA dissolved in 50 microliters of distilled water was added to the flask. The solution was refluxed for 2 hours, after which the acetone was evaporated. The residual solid was extracted with ether and water and the aqueous layer discarded. The extractate of the ether layer was evaporated producing the cholesteryl ester of GABA. The ester was dissolved in 200 μl of diethyl ether, 200 μl of methanol and 200 μl of a hydroxylamine solution that was previously prepared by combining equal volumes of 5% hydroxylamine HCL and 12.5% sodium hydroxide and filtering the sodium chloride precipitate. (Thompson, 1950) The solution was placed in a water bath at 45 degrees centigrade for 30 minutes and then 1 ml of ferric perchlorate reagent solution was added. A pink-purple color indicated the presence of the cholesteryl ester of GABA. (FIG. 2)

### Synthesis of the Cholesteryl Ester of Leu-Enkephalin

2. Ten microliters of 1.0 M hydrochloric acid was dissolved in a solution of 25 ml of acetone containing 0.016 millimoles or 6 mg of cholesterol. A solution containing 0.05 millimoles or 25 mg of leu-enkephalin (Genscript, Grand Cayman, Cayman Islands) was dissolved in 50 microliters of distilled water and added to the flask. The solution was refluxed for 2 hours after which the acetone was evaporated. The residual solid was extracted with ether and water and the aqueous layer discarded. The extractate of the ether layer was evaporated producing the cholesteryl ester of leu-enkephalin. The cholesteryl ester of leu-enkephalin was dissolved in 200 μl of diethyl ether, 200 μl of methanol and 200 μl of a hydroxylamine solution. The solution was placed in a water bath at 45 degrees centigrade for 30 minutes and then 1 ml of ferric perchlorate reagent solution was added. A pink-purple color change indicated the presence of the cholesteryl ester of leu-enkephalin. (FIG. 2)

### Synthesis of Cholesteryl Ester of (PLLA)

3. One hundred milligrams of L-lactic acid was polymerized in a microwave to 80 mg of PLLA with loss of 20 mg of water. The PLLA was dissolved in 20 microliters of water. Ten microliters of 1.0 M hydrochloric acid was dissolved in a solution of 25 ml of acetone containing 0.25 millimoles or 96 mg of cholesterol. An aqueous solution containing 80 mg of PLLA in 20 microliters of water was added to flask. The solution was refluxed for 2 hours after which the acetone was evaporated. The residual solid was extracted with ether and water and the aqueous layer discarded. The extractate of the ether layer was evaporated producing the cholesteryl ester of PLLA. The ester was dissolved in 200 μl of diethyl ether, 200 μl of methanol and 200 μl of a hydroxylamine solution. The solution was placed in a water bath at 45 degrees centigrade for 30 minutes and then 1 ml of ferric perchlorate reagent solution was added. A pink-purple color change indicated the presence of the cholesteryl ester of PLLA. (FIG. 2)

### Controls

A. 200 μl of hydroxylamine solution was added to 200 μl of diethyl ether and 200 pi of methanol and placed in a water bath at 45 degrees centigrade for 30 minutes. 1 ml of ferric perchlorate reagent solution was added. A yellow-amber color change indicated no evidence of ester.

B. 10 mg GABA, 10 mg leu-enkephalin, 10 mg PLLA and 10 mg cholesterol, were each dissolved in 200 μl of methanol 200 μl ether and 200 μl of hydroxylamine solution and placed in a water bath at 45 degrees centigrade for 30 minutes. Addition of of ferric perchlorate reagent solution produced a yellow-amber color.

### References

Abuilaiwi, F. A., Laoui, T., Al-Harthi, M., & Atieh, M. A. (2010). Modification and Functionalization of Multiwalled Carbon Nanotube (Mwcnt) Via Fischer Esterification. Arabian Journal for Science and Engineering, 35 (1c), 37-48.

Cole, A. C., Jensen, J. L., Ntai, I., Tran, K. L., Weaver, K. J., Forbes, D. C., & Davis, J. H., Jr. (2002). Novel Bronsted acidic ionic liquids and their use as dual solvent-catalysts. J Am Chem Soc, 124 (21), 5962-5963.

Forbes, D. C., & Weaver, K. J. (2004). Bronsted acidic ionic liquids: the dependence on water of the Fischer esterification of acetic acid and ethanol. Journal of Molecular Catalysis a-Chemical, 214 (1), 129-132. doi: 10.1016/j.molcata.2003.09.035

Goldberg, J. S., Weinberg, J. B. (2016). U.S. Pat. No. 9,382,376B2.USPTO.

Jacob, J. N., Hesse, G. W., & Shashoua, V. E. (1987). gamma-Aminobutyric acid esters. 3. Synthesis, brain uptake, and pharmacological properties of C-18 glyceryl lipid esters of GABA with varying degree of unsaturation.

J Med Chem, 30 (9), 1573-1576.

Jacob, J. N., Hesse, G. W., & Shashoua, V. E. (1990). Synthesis, brain uptake, and pharmacological properties of a glyceryl lipid containing GABA and the GABA-T inhibitor gamma-vinyl-GABA. J Med Chem, 33 (2), 733-736.

Joseph, T., Sahoo, S., & Halligudi, S. B. (2005). Bronsted acidic ionic liquids: A green, efficient and reusable catalyst system and reaction medium for Fischer esterification. Journal of Molecular Catalysis a-Chemical, 234 (1-2), 107-110. del: 10.1016/j.molcata.2005.03.005

Shashoua, Victor E. (1991). U.S. Pat. No. 5,051,448. USPTO.

Spinella, S. M., Maiorana, A., Lo Re, G., Raquez, J. M., Dubois, P., & Gross, R. A. (2014). Cellulose nanocrystals decorated with free carboxylic acids by one step hydrolysis/Fischer esterification reactions. Abstracts of Papers of the American Chemical Society, 248.

Spinella, S., Maiorana, A., Qian, Q., Dawson, N. J., Hepworth, V., McCallum, S. A., . . . Gross, R. A. (2016). Concurrent Cellulose Hydrolysis and Esterification to Prepare a Surface-Modified Cellulose Nanocrystal Decorated with Carboxylic Acid Moieties. Acs Sustainable Chemistry & Engineering, 4 (3), 1538-1550. del: 10.1021/acssuschemeng.5b01489

Thompson, Adrienne R. (1950). A colorimetric method for the determination of esters. Australian Journal of Chemistry, 3.1, 128-135.

Having described my invention, I claim:

1. A modification of the Fischer esterification reaction for the preparation of the cholesteryl ester of leu-enkephalin comprising:
a) refluxing cholesterol and leu-enkephalin in molar ratios of 1:2 to 1:4 in acetone that has been acidified with 1.0 M hydrochloric acid to a pH of 1-4
b) evaporating the acetone to yield a solid substance
c) dissolving the solid substance in diethyl ether and water
d) extracting the solid substance with diethyl ether and water and
e) evaporating the diethyl ether solution to obtain the cholesteryl ester of leu-enkephalin.

2. The method of claim 1 for the preparation of the cholesteryl ester of poly D-lactic acid or poly L-lactic acid.

3. A modification of the Fischer esterification reaction comprising:
a) refluxing heterogeneous solutions of a carboxylic acid and a primary or secondary alcohol where one reagent is lipophilic and the other reagent is hydrophilic
b) refluxing without the use of chemical scaffolds or acidic ionic liquids.
